(19) United States
(12) Patent Application Publication
(10) Pub. No.: US 2011/0070636 A1
(43) Pub. Date: Mar. 24, 2011

(54) RESILIENT MEMBER AND DEVICE, IN PARTICULAR A BIO FILM REACTOR

(75) Inventor: Anders Dalsgaard, Viborg (DK)

(73) Assignee: GRUNDFOS BIOBOOSTER A/S, Bjerringbro (DK)

(21) Appl. No.: 12/991,680

(22) PCT Filed: May 18, 2009
(86) PCT No.: PCT/DK2009/000112

§ 371 Date: Dec. 6, 2010

(30) Foreign Application Priority Data
May 16, 2008 (EP) 08103997.6

(51) Int. Cl. C12M 1/14

(52) U.S. Cl. 435/298.1

(57) ABSTRACT

The present invention relates to a resilient member (FIG. 7b) and combination of resilient members into resilient devices. Particular embodiments of the present invention relate to a bio film reactor comprising a stack of bio film discs formed as a resilient member thereby allowing the stack of bio film discs to be self compensative at least partially for expansion of the stack as a result of e.g. changes in temperature. Furthermore, the resilient member and device according to the present invention provide a great flexibility as to the actual choice of material for the member and device. The male protrusions (4) of one resilient member (1) are introduced into the voids (5) of the female protrusions (3) of a similar or identical member (1). Male protrusions (4) of a second kind do not abut the bottom of the voids (5).

### FIELD OF THE INVENTION

The present invention relates to a resilient member and combination of resilient members into resilient devices. Particular embodiments of the present invention relate to a bio film reactor comprising a stack of bio film discs formed as a resilient member thereby allowing the stack of bio film discs to be self-compensative at least partially for expansion of the stack as a result of e.g. changes in temperature. Furthermore, the resilient member and device according to the present invention provide a great flexibility as to the actual choice of material for the member and device.

### BACKGROUND OF THE INVENTION

Many constructions are designed to operate in and/or being stored at varying climate conditions. Typically, the changes in climate include changes in temperature, humidity, pressure and such changes will in many cases result in the dimensions of the construction being changed as the material of the construction may expand or shrink as a result of the change in climate.

Often such changes in dimensions are compensated by one or more resilient members typically being springs, expansion joint or the like. As such expansions or shrinking represent one or more geometrical freedom in the construction, many such constructions also need a member to keep the elements assembled during expansion and/or shrinking.

A resilient member is typically build into a construction only to compensate for geometrically changes in the construction and does not contribute to the primary operation of the construction but renders it possible for the construction to operate at different design conditions. Hereby such resilient member may represent an unwanted space occupying element.

### OBJECT OF THE INVENTION

While many of the known resilient members (springs, expansion joints etc) allowing a construction to expand and/or shrink works effectively, they often take up much place in the construction as they often are separate parts of a construction. Furthermore, many of the known resilient members only contribute to keep the constructions assembled by it resiliency whereby further elements often are need to keep the various part of the construction assembled.

It is an object of the present invention to provide a resilient member and/or resilient device, in particular a bio film reactor at least mitigating some of the drawbacks encountered by springs, expansion joint and the like.

### SUMMARY OF THE INVENTION

Thus, the present invention relates in a first aspect to a resilient member. The resilient member is preferably a bio film disc. Such bio film discs are typically stacked and one or more such stacks comprising similar or identical resilient members are arranged within a bio film reactor. The resilient member and in particular the bio film discs comprising a base part having male protrusions and voids wherein
the voids are adapted to receive male protrusions of a similar or identical resilient member so as to define a resilient device in which
   a part of a first number of male protrusions abuts one or more parts of the similar or identical resilient member when introduced into voids thereby hindering distal ends of said number of male protrusions from penetrating further into the voids and
   in which the distal end of a second number of the male protrusions may penetrate further into said voids by elastic deformation of the base part when the distal ends of the first number of male protrusions are hindered from penetrating further into voids.

In the present disclosure, a number of terms are used. Although these terms are used in a manner ordinary to a person skilled in the art, a brief explanation of some of these terms will be presented below.

A resilient member according to the present invention is accordingly adapted to co-operate with a similar or identical member to define a resilient device. By “similar” is preferably meant a member which comprises protrusions and voids which may co-operate with the member in question. By “ identical” is preferably meant a member being identical i.e. having the same arrangement of male protrusions, female protrusions and voids, to the member in question. Accordingly, identical is preferably used to quantify that two or more elements are produced in the same manner by a given manufacturing process so that the elements have the same geometrical characteristics and dimensions within the production tolerances available by the tools used. However, identical elements may vary in colour, shading and the like when not being a relevant measure to consider due to the use of the elements.

Disc is used to designate not necessarily a circular structure; accordingly, a disc may have any type of shaped perimeter such as circular, square, triangular, rectangular, oval, ellipsoid and polygon or similar. Consequently, the discs may be described as plate shaped wherein plate does not necessarily imply that the disc is made of metal: Accordingly, the disc can be made of any type of material such as metal, plastics or similar.

In accordance with the invention, the resiliency stems mainly from deformation of the base part and the material of the base part is selected so that the deformations are only elastic deformations so that the resilient member and device keeps its originally produced shape when no load is applied.

Preferred embodiments of the resilient member preferably comprises a number of first female protrusions having a first longitudinal extension and a number of second female protrusions having a second longitudinal extension being shorter than the first longitudinal extension, wherein the first and the second female protrusions each comprises one of said voids.

The male protrusions and/or the female protrusions, if present, may extend in normal direction to the base part.

In some embodiments of the invention, the male protrusions extend in a direction being opposite to the direction in which the female protrusions extend, and the male protrusions may be of equal length. The voids of the female protrusions may in addition or alternatively be adapted to fully receive the male protrusions.

In combination thereto, the resilient member may further comprise a number of female protrusions in which said voids are arranged, wherein the female protrusions extend in a direction being opposite to the direction in which the male protrusions extend and wherein the female protrusions are of equal length.

In preferred embodiments of the invention, the male protrusions may comprise a number of first male protrusions having a first longitudinal extension and a number of second male protrusions having a second longitudinal extension being shorter than the first longitudinal extension.

In some embodiments of the invention, all the male and the female protrusions may point in the same direction and the male protrusions may be provided as extensions to the female protrusions.

In alternative embodiments of the invention, the voids may be provided in the base part.

The voids may preferably comprise a bottom so that the distal ends of a number of the male protrusions may abut said bottom so as to hinder said number of male protrusion to penetrate further into said voids.

A further aspect of the present invention relates to a resilient member being a bio film disc for a bio film reactor preferably being a pressurised bio film reactor. The bio film disc may preferably comprise one or more of the features of the resilient member according to the present invention.

Preferably, the bio film disc comprises a rim along which the male protrusions, the female protrusion, if present, and the voids are arranged.

In still a further aspect, the invention relates to a bio film reactor comprising one or more stacks of bio film discs according to the present invention.

Further embodiments and aspects of the invention are present in the claims as well as in the following description.

### BRIEF DESCRIPTION OF THE FIGURES

The present invention and in particular preferred embodiments thereof will now be disclosed in connection with the accompanying figures in which:

FIG. 1 shows schematically a first embodiment of a resilient member according to the present invention,

FIG. 2 shows schematically a resilient device assembled from resilient members shown in FIG. 1,

FIG. 3 shows schematically deformation of a resilient device assembled from resilient members according to the present invention,

FIG. 4 shows schematically a further embodiment of a resilient member according to the present invention,

FIG. 5 shows schematically various embodiments of resilient members according to the present invention,

FIG. 6 shows schematically a pressurised bio film reactor according to a preferred embodiment of the present invention,

FIG. 7 shows schematically protrusions of the bio film discs so as to incorporate resiliency to the bio film disc,

FIG. 8 shows in details a bio film disc according to the present invention,

FIG. 9 shows schematically changes in longitudinal direction of a disc stack.

### DETAILED DESCRIPTION OF PREFERRED EMBODIMENTS OF THE PRESENT INVENTION

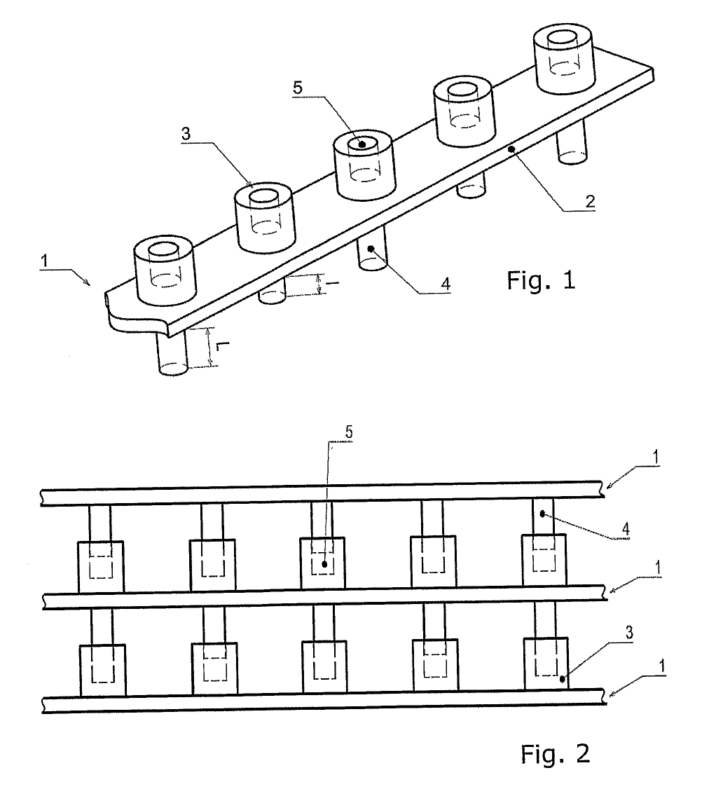

FIG. 1 shows schematically a first embodiment of a resilient member 1 according to the present invention. The resilient member 1 comprises a base part 2 (in FIG. 1, the curved end indicates that the length of the base part 2 and thus the resilient member may be longer or shorter than indicated in the figure). On one side of the base part 2 female protrusions 3 are provided and on the other side corresponding male protrusions 4 are provided. The male protrusions 4 are typically cylindrically shaped with equal diameter. However, the male protrusions 4 may be given other shapes deviating from cylindrical and the diameter—or in general their thickness—may vary both along the longitudinal direction of the male protrusion 4 and between the male protrusions 4 themselves as long as they fit into corresponding female protrusion 3 as will be disclosed below. The male protrusions 4 are of two different lengths, first protrusions of length L and second protrusions of length I, wherein the first protrusions are longer than the second protrusions, as indicated in the figure. The male protrusions 4 are distributed on the base part 2 so that two of the first male protrusions are not located adjacent to each other. Typical and preferred distributions of the male protrusions 4 are L, I, L, I, L; L, I, I, L, I, I, L and L, I, I, I, L, I, I, I, L, i.e. one, two or three second protrusions between two first protrusions. In the embodiment shown in FIG. 1, the male protrusion located end the end of the member (as seen from the end located to the right in the figure) is of length L and the sequence shown is L, I, L, I etc. However, the sequence may be I, L, I, L etc or another sequence as disclosed above. Thus, the member comprising a first number of male protrusions each with length I and a second number of male protrusions each with a length L. Typically and preferably, no other male (or in case of female protrusions) are provided.

The resilient member 1 is used in combination with one or more similar or identical member as illustrated in FIG. 2 where three resilient members 1 are combined to form a resilient device (please note that the female protrusions on the upper resilient member and the male protrusion on the lower resilient member is left out). As shown in the figure, the male protrusions 4 of one resilient member 1 are introduced into the voids 5 of the female protrusions 3 of a similar or identical resilient member 1 to such an extent that the distal ends of the first protrusions abut the bottom of the voids 5 of the female protrusions 3. Furthermore, the second male protrusions are also introduced into the voids 5 of the female protrusions 3. However, as the distal ends of the second male protrusions 4 do not abut the bottom of the voids 5 of the female protrusions 3, the second male protrusion are able to be introduced further into the voids 5 of the female protrusions 3. As the base part 2 is made from a resilient material, a bending of the base part 2 can allow the second male protrusion to penetrate further into the voids of female protrusions until the distal ends of the second protrusions abut the bottom of the voids 5. The bending may be provided by a loading e.g. a force applied to the resilient members 1 in a direction parallel to the longitudinal direction of the male protrusions 4.

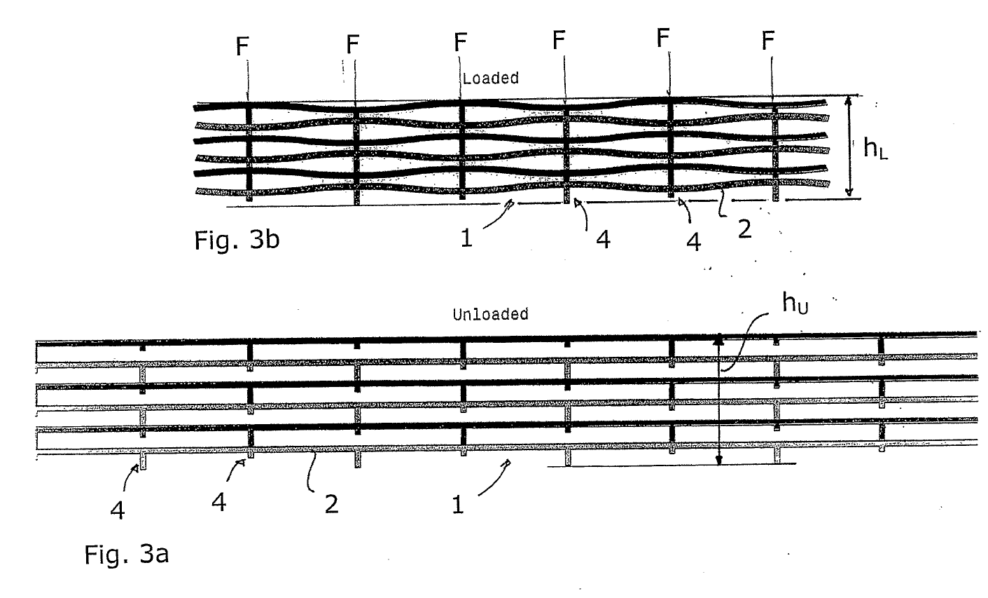

Such a situation is disclosed schematically in FIG. 3 which shows a cut out of a stack of resilient members (six resilient members 1 are shown in the figure). In the figure, the female protrusions 3 are left out and different shadings have been applied to the resilient members 1 to render the figure clearer.

The resilient member 1 showed on top of the stack is an end resilient member 1 with no female protrusions 3 so as to form e.g. an abutment surface for abutting a surface of a construction in which the stack of resilient members is used; a similar end resilient member with no male protrusions is typically applied at the opposite end of the stack.

25 FIG. 3a shows the stack of resilient members 1 in unloaded state when no external force has been applied to the stack in a direction normal to the plane defined by the base part 2. FIG. 3b shows the same stack of resilient members 1 as shown in FIG. 3a but in a loaded state where an evenly distributed force F has been applied to the stack in a direction normal to the plane defined by the base part 2 on the abutment surface of the end resilient member 1 as indicated in FIG. 3b. As shown in FIG. 3b, the stack of resilient members 1 is compressed as a result of the force F, and the compression is a result of the deformation of the base parts 2 which each deforms into a curved shaped as indicated in FIG. 3b. The stack may be compressed until the distal ends of the second male protrusions 4 abut the bottom of the voids 5 of the female protrusions 3 which in FIG. 3b is indicated by the distal ends of the second male protrusions abutting the base part 2 as the female protrusions are left out in the figure. As indicated in FIG. 3, h_{L} (height in loaded state) is different to h_{U} (height in unloaded state), in particular, h_{L} is smaller than h_{U}.

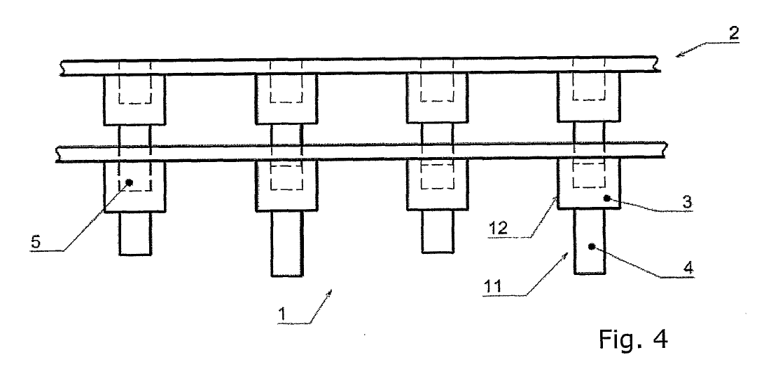

FIG. 4 shows an alternative embodiment of the resilient member according to the present invention. In this embodiment, male protrusions 4 are provided on one side of the base member 2 similarly to what was disclosed in connection with FIGS. 1-3. However, female protrusions are provided on the base part 2 on the same side as the male protrusions 4 and each comprises a void 5 adapted to receive male protrusions 4 of an adjacent resilient member 1. Accordingly, the male protrusions 4 are provided as an extension to the female protrusions so that a protrusion comprising two sections of different size is provided on the base part 2: an outer section 11 forming a male protrusion adapted enter into a void 5 and an inner section 12 forming a female protrusion adapted to comprise a void 5. Typically, the male protrusions and female protrusions are cylindrically shaped with two different diameters as shown in the figure. Also in this embodiment, the longer male protrusions abut the bottom of the voids while the shorter male protrusions are able to penetrate further into the voids. As disclosed in connection with FIG. 1 the male and female protrusions may be given other shapes.

In an alternative embodiment (not shown) of the embodiment shown in FIG. 4, no inner sections 12 is provided and the resilient member 1 thus does not comprise female protrusions. Instead, the male protrusions 4 are of two different lengths and the base part 2 comprises voids with a bottom into which the male protrusions 4 extend. When two or more resilient members are combined to form a resilient device, the longer male protrusions (first protrusions) extend into the voids and the distal ends of the first male protrusions abut the bottom of the voids. Furthermore, the shorter male protrusions (second protrusions) are able to penetrate further into the voids by deformation of the base part 2 until the distal ends of the second male protrusions abut the bottom of the voids.

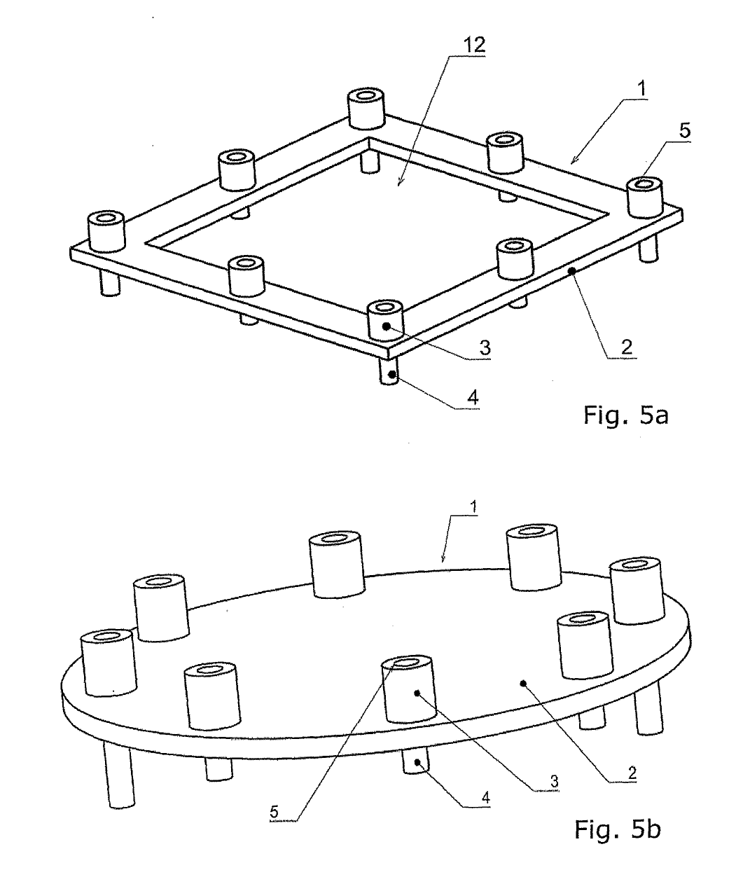

The resilient members 1 shown in FIGS. 1-4 are all shown as elongated members. However, the resilient members 1 may be produced in other shapes of which a few are disclosed in FIG. 5.

FIG. 5a shows a square shaped resilient member 1. A square shaped cut out 13 is provided in the base part and a number of male and female protrusions with voids are provided on the base part 2 similarly to what was disclosed in connection with FIGS. 1-4. The square shaped cut out 13 may be omitted. The number of male and female protrusions may vary and only few are shown in FIG. 5a.

FIG. 5b shows a circular shaped resilient member 1 with a number of male and female protrusions 4, 3 provided on the base part 2 similarly to what has been disclosed in connection with FIGS. 1-4. Also in this embodiment the actual number of male and female protrusions may vary and only a few are shown in FIG. 5b. The male and female protrusions are arranged along the rim of the resilient member. Although FIG. 5b may give the impression that the male and female protrusions are not evenly distributed along the rim, this is due to the perspective view in the figure and the protrusions are preferably evenly disturbed along the rim. However, embodiments where such an even distribution is not present is within the scope of the present invention.

The various configurations of male and female protrusions disclosed in connection with FIGS. 1-4 may advantageously be applied to the members disclosed in FIG. 5 as well as to the following disclosure pertaining to a bio film disc.

The resilient member according to the present invention may be produced from a number of materials. Preferably, the resilient member is produced from plastic, metal or the like.

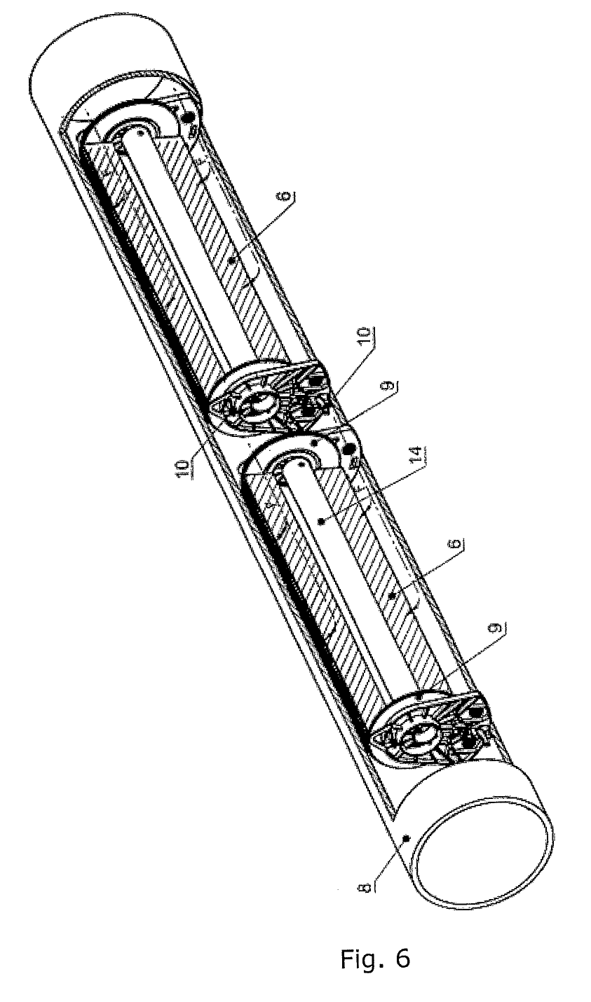

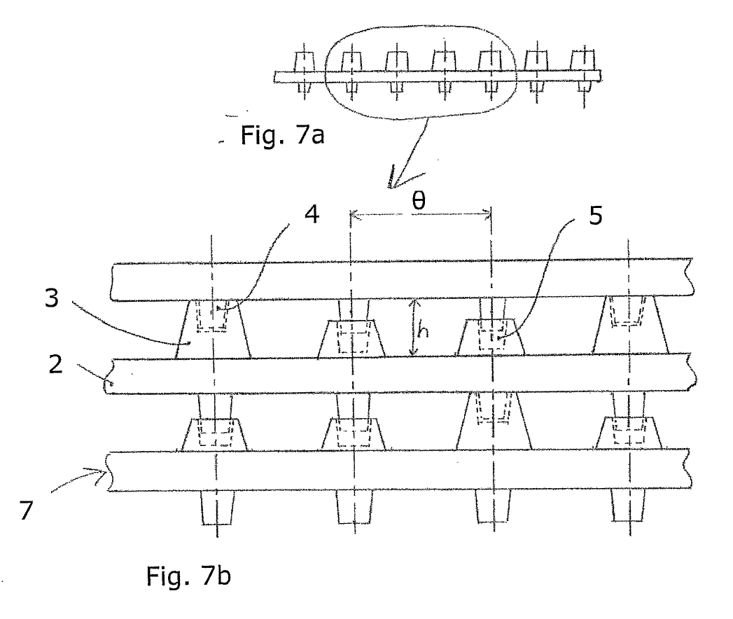

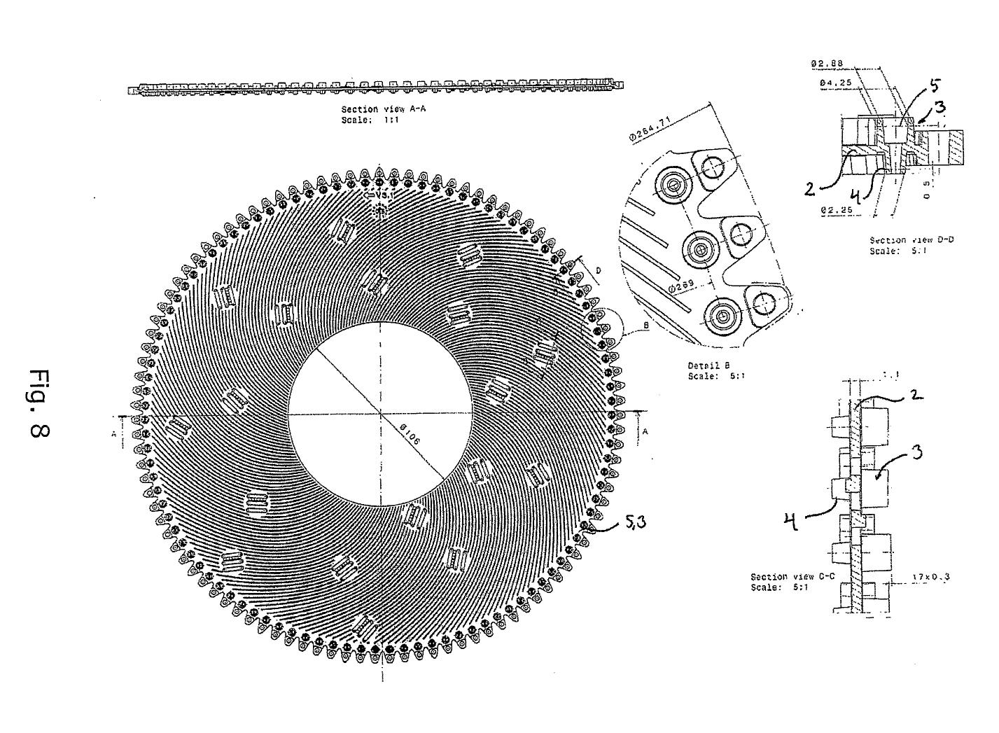

A resilient member 1 according to the present invention has been found particularly useful in connection with a so called pressurised bio film reactor which is schematically disclosed in FIG. 6. The bio film reactor comprises one or more wagons 6 each comprising a stack of discs being disposed inside a tubular casing 8 (a part of which is disclosed in FIG. 6). The stack of discs is arranged on a central shaft 14 on which end elements 9 are fixedly mounted. The discs 7 in a wagon 6 are typically pretensioned between two end elements 9 but able to move along the central shaft between the end elements 9. The wagons 6 are assembled by clips 10 which may be integrated in the end elements 9. Fluid to be treated in the reactor flows between the discs and a bio film is generated between the discs. FIG. 7a shows a bio film disc according to the present invention (see also FIG. 8 which shows further details on the bio film disc 7) and FIG. 7b shows in a close up view the protrusions provided on the rim of the disc of FIG. 7a (in FIG. 7b three discs are shown stacked and the female protrusions on the upper disc is omitted). As shown in FIG. 7, each of the bio film discs 7 comprises protrusions similar to what has been disclosed in connection with FIGS. 1-5, in particular 5a and 5b, and extending in a direction normal to the plane of the disc 7 along the rim of the disc 7 on both sides of the discs 7. The protrusions on one side of the disc are male protrusions 4, and the protrusions on the other side of the disc 7 are female protrusions 3 with voids 5 so that when two discs 7 are stacked on top of each other, the protrusions engage as disclosed in FIG. 7b. The protrusions shown in FIG. 7 represent a further embodiment of the resilient member according to the present invention. The female protrusions 3 in this embodiment still comprise voids, but the female protrusions are given two different lengths and the male protrusions are of equal length. This may be characterised as opposite to the embodiment disclosed in FIG. 1 in which the male protrusions are of two different lengths. The voids 5 and the male protrusions 4 are shaped so that a first number of male protrusions 4 are fully received in the first female protrusions 3 (the longer ones in FIG. 7b) whereby the distal ends of the female protrusions abut the base part 2 as shown in FIG. 7b. A second number of male protrusions 4 may penetrate further into second female protrusions 3 (the short ones in FIG. 7b) by elastic deformation of the base part 2.

As shown in FIG. 7b, the stacking of the discs 7 results in a spacing h between the discs 7. As the protrusions are spaced a distance θ from each other in circumferential direction, an opening is provided between the discs 7 so that fluid may flow into the space between the discs 7.

Fluid to be treated by the bio film reactor flows into the reactor through an end element 9 and flows along the inner side of the casing 8 and into the space between the bio film discs 7 as indicated by the arrows labelled F in FIG. 6.

A wagon 6 typically comprises up to 250 bio film discs 7. It typically operates at a temperature around +25° C. and is stored at temperatures varying typically from −20° C. to +80° C. In this connection it has been found that the length of a wagon can vary up to around 10% in such a temperature range if no resiliency is present in the wagon to compensate for the expansion of the discs 7. If no such resiliency is present, the increase in length must be compensated for by allowing the wagon to expand inside the casing which leads to a lower utilisation of the volume inside the casing 8.

Resiliency is applied to the stack of discs by the male and female protrusions. As shown in FIG. 7 and corresponding to what was disclosed above for male protrusions 4 having different lengths, the female protrusions 3 have two different lengths, L and I, whereas the male protrusions 4 are of equal length. Thus, when bio film discs are stacked, the male protrusions 4 are able to penetrate further into the voids 5 of the short female protrusions thereby providing a resiliency to the stack of discs which are able to compensate for expansion of the stack of discs during temperature changes.

The bio film discs 7 are preferably orderly stacked so that two adjacent bio film discs 7 are rotated relatively to each other so that two first female protrusions 3 with length L are not located at the same angular position on the rim of the two adjacent discs in question. However, it has been found in connection with the present invention that casual stacking of the discs (without paying attention to the location of the first female protrusion) often result in a stacking which has substantially the same resilient effect as when the bio film discs 7 are orderly stacked.

The casual stacking may advantageously be applied to the members disclosed in connection with the FIGS. 1-5.

The presence of the first female protrusion 3 in combination with the elasticity of the bio film discs 7 will provide a resiliency to the stack which results in a buckling of the discs 7 if a force is applied to the stack of discs in the longitudinal direction of the discs 7. Such as force will be generated when the discs 7 expand and the length of the stack of discs is fixed. Thus, in a preferred embodiment the distance between wagons 6 may be minimised and in some embodiment totally avoided. In the latter case, the discs 7 are not considered as being stacked in wagons but the discs 7 inside the casing 8 are considered to be stacked in a single stack.

FIG. 8 shows an embodiment of a bio film disc 7 according to the present invention (dimensions in the figure is given in mm). The bio film disc 7 is similar to what has been disclosed in connection with FIG. 7 although further details are provided in FIG. 8. Similar features as disclosed in FIG. 7 are referenced in FIG. 8 by the same reference numbers. The bio film disc 7 shown in FIG. 8 has a thickness of 1.1 mm inside the rim comprising teeth and the male protrusions 4 and the female protrusions 3. The actual shaping of the male and the female protrusions 4, 3 are indicated in section view D-D of FIG. 8.

Section view C-C of FIG. 8 shows the difference in height of the female protrusions 3. The male protrusions 4 are shaped as truncated cones. However, they may alternatively be cylindrically shaped in which case the voids are given a corresponding shape. As shown, there is a 0.3 mm difference in height which allows male protrusion to penetrate further into the void 5 of the shorter female protrusion 3 when two discs are arranged on top of each other and the distal ends of the female protrusions 3 abut the base part 2.

Sections views C-C and D-D also show the coning of the protrusions. The coning makes the protrusions easier to manufacture and assist in guiding the male protrusions 4 into corresponding female protrusions 3.

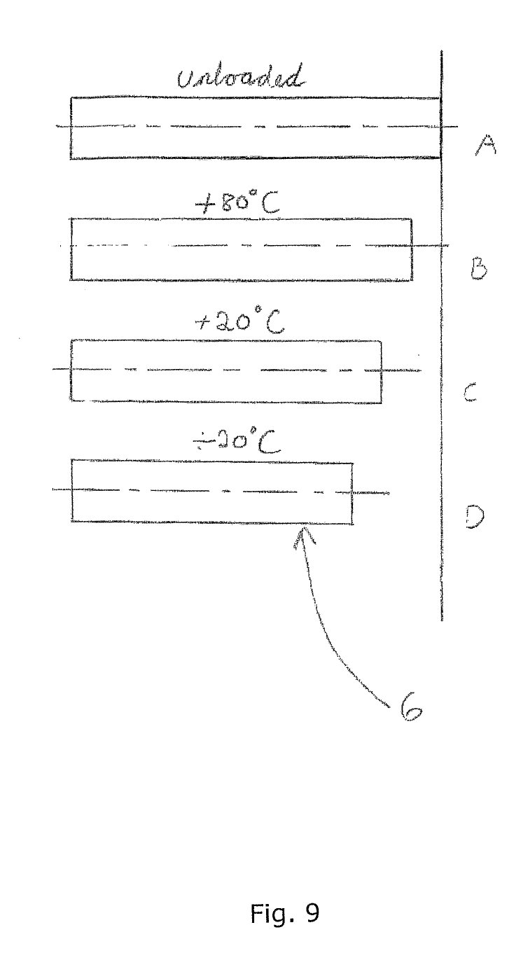

FIG. 9 shows schematically the changes in longitudinal extension of a wagon 6. When the discs are stacked and no pretension is applied to the stack, the length of the stack is 1095.55 mm as shown in FIG. 9a. FIGS. 9b-d shows the longitudinal extension of the stack of discs at three different temperature levels. The stack of discs is typically designed to operate at a temperature between +15° C. and +40° C., such as around +25° C., and being stored and cleaned between −20° C. and +80° C. However, other operating, storing and cleaning temperatures are also possible within the scope of the present invention. In order to keep the stack assembled in this temperature interval, the stack is compressed by the end elements 9 so that the stack is kept assembled at the lowest temperature which is shown in FIG. 9d.

Considering a situation where the temperature of the stack is initially −20° C., the bio film discs expand as the temperature increases, and as the bio film discs 7 are resilient members, some of the expansion will be compensated by a buckling of the discs 7 whereby the need for larger compression by the end elements 9 is mitigated.

FIG. 3 is representative for the buckling of the bio film discs 7 as seen from the outside of the discs in a direction parallel to the radius of the discs; please note that the embodiment shown in e.g. FIG. 1 has been applied in this case (contrary to what is disclosed in connection with FIG. 7b) and that the female protrusions 3 are left out for clarity reasons. Furthermore, different shadings are provided to the discs although the discs are identical to each other. FIG. 3b represents in this case a stack of six bio film discs 7 (only a cut-out is shown) in a loaded situation where no further compression of the stack is possible without compressing the discs and the protrusions themselves. FIG. 3a shows the same stack in an unloaded situation.

1. A bio film reactor comprising one or more stacks of similar or identical resilient members, wherein the resilient members are bio film discs, each comprising:
a base part having male protrusions and voids; and
a rim along which the male protrusions and the voids are arranged, wherein
the voids are adapted to receive male protrusions of a similar or identical resilient member so as to define a resilient device in which:
a part of a first number of male protrusions abuts one or more parts of the similar or identical resilient member when introduced into voids thereby hindering distal ends of said number of male protrusions from penetrating further into the voids, and
in which the distal end of a second number of the male protrusions may penetrate further into voids by elastic deformation of the base part when the distal ends of the first number of male protrusions are hindered from penetrating further into the voids.

2. The bio film reactor according to claim 1, wherein the rim of the bio film disc further comprises a number of first female protrusions having a first longitudinal extension and a number of second female protrusions having a second longitudinal extension being shorter than the first longitudinal extension, wherein the first and the second female protrusions each comprises one of said voids.

3. A The bio film reactor according to claim 1, wherein the male protrusions or the female protrusions, if present, extend in normal direction to the base part.

4. A The bio film reactor according to claim 2, wherein the male protrusions extend in a direction being opposite to the direction in which the female protrusions extend and, wherein the male protrusions are of equal length.

5. A The bio film reactor according to claim 2, wherein voids of the female protrusions are adapted to fully receive the male protrusions.

6. The bio film reactor according to claim 1, wherein the rim of the bio film disc further comprises a number of female protrusions in which said voids are arranged, wherein the female protrusions extend in a direction being opposite to the direction in which the male protrusions extend and, wherein the female protrusions are of equal length.

7. The bio film reactor according to claim 1, wherein the male protrusions comprise a number of first male protrusions having a first longitudinal extension and a number of second male protrusions having a second longitudinal extension being shorter than the first longitudinal extension.

8. The bio film reactor according to claim 2, wherein all the male and the female protrusions point in the same direction and, wherein the male protrusions are provided as extensions to the female protrusions.

9. The bio film reactor according to claim 1, wherein the voids are provided in the base part.

10. The bio film reactor according to claim 1, wherein said voids comprise a bottom so that the distal ends of the first number of the male protrusions may abut said bottom so as to hinder said number of male protrusion to penetrate further into said voids.

11. The bio film reactor according to claim 1, wherein the bio film disc is made from an elastic material.

12. The bio film reactor according to claim 1, wherein no further male protrusions than the first and second number of male protrusions are arranged along the rim and that no further voids are arranged than a number corresponding to the total number of male protrusions.

13. (canceled)
